(19) United States
(12) Patent Application Publication
(10) Pub. No.: US 2025/0288984 A1
(43) Pub. Date: Sep. 18, 2025

(54) FUNCTIONAL ELEMENT, BETA CONTAINER SYSTEM, TRANSFER SYSTEM AND BARRIER SYSTEM

(71) Applicant: Groninger & Co. GmbH, Crailsheim (DE)

(72) Inventors: Armin MERZ, Ellwangen (DE); Matthias SCHNEIDER, Stimpfach-Rechenberg (DE)

(21) Appl. No.: 19/222,099

(22) Filed: May 29, 2025

Related U.S. Application Data
(63) Continuation of application No. PCT/EP2023/083515, filed on Nov. 29, 2023.

(30) Foreign Application Priority Data
Nov. 30, 2022 (DE) 10 2022 131 808.7

(51) Int. Cl. B01L 1/02; B65G 11/02

(52) U.S. Cl. CPC B01L 1/025; B65G 11/02

(57) ABSTRACT

A functional element for a beta container system for transferring objects, wherein the functional element has a transfer surface for transferring the objects through a beta port opening of a beta port of a beta container of the beta container system. The functional element can be arranged on the beta container in a defined arrangement, the transfer surface is arranged at least partially in the beta port opening in the defined arrangement, and a first bag section of a bag of the beta container is arranged in the defined arrangement between the beta port and the functional element. Also disclosed is a beta container system, transfer systems and a barrier system.

### CROSS-REFERENCE TO RELATED APPLICATIONS

This is a Continuation application of International patent application PCT/EP2023/083515, filed Nov. 29, 2023, which claims the priority of German patent application DE 10 2022 131 808.7 filed Nov. 30, 2022. Both application PCT/EP2023/083515 and DE 10 2022 131 808.7 are herewith incorporated by reference in their entireties.

### FIELD

The present invention generally relates to a functional element for a beta container system for transferring objects, to a beta container system for a transfer system, to a transfer system, and to a barrier system, in particular an isolator system.

### BACKGROUND

A barrier system is a system that provides a physical and aerodynamic barrier, e.g. by means of positive air pressure, between an external environment, for example an external cleanroom environment, and a work process. Various barrier systems are known in the prior art. A barrier system can, for example, be an isolator or a barrier with restricted access, a so-called RABS (Restricted Area Barrier System). The RABS can be an open RABS or a closed RABS.

The present disclosure is primarily concerned with aseptic isolators as barrier systems. However, the present disclosure can also be used in other barrier systems.

The term “isolator” generally refers to a container that is hermetically and gas-tightly sealed from the surrounding work space. A defined atmosphere can be created within an isolator for processing sensitive or dangerous products.

In this context, isolators are typically used in biopharmaceutical process engineering, for example as part of a filling system with multiple process and processing stations, to create a highly clean or sterile, i.e. germ-free, environment.

In such filling systems, for example, containers, in particular vials, cartridges, bottles, syringes and/or the like, can be filled with a product, preferably a pharmaceutical or cosmetic product, in particular a liquid or a powder, and then closed with a closure element, for example a stopper or a closure cap, in particular a crimp cap.

Transfer systems can be used to transfer objects, such as closure elements, into or out of the isolator. A transfer system may have a transfer lock that can be coupled to the isolator from the outside. Objects can be transferred into or out of the isolator via the transfer lock. A transfer system can, for example, be designed as a port system, in particular as an alpha-beta port system. Such transfer systems have an alpha port and a beta container. The beta container serves as a transfer lock. The beta container has a beta port. The alpha port is arranged preferably on an isolator wall of the isolator which surrounds an interior space of the isolator. The alpha port and the beta port can be coupled together to connect the interior space of the isolator to an internal volume of the beta container. In the coupled state, objects can be brought from the beta container into the isolator or objects can be brought from the isolator into the beta container.

Such beta containers may, for example, have a bag that encloses the internal volume of the beta container. In particular, a support structure, such as a carriage, with a support surface for the bag may be provided outside the isolator in order to position the bag accordingly relative to the beta port.

Feeding devices can be used to feed closure elements provided in a beta container. A feeding device can be arranged within the isolator and can be arranged from the inside on the alpha port. The feed device can, for example, comprise a chute or a pipe. The closure elements can then be inserted or fed from the beta container into the isolator via the feed devices.

For example, the document DE 10 2021 101 384 B3 shows a system for transporting sterile closure elements, suitable for pouring, from surroundings of an isolator into an interior space of the isolator, comprising a container for storing a supply quantity of closure elements in the surroundings of the isolator, an isolator opening and a collecting device for collecting the closure elements and for providing the closure elements in the interior space of the isolator, with a dosing device for controlling a desired quantity of closure elements to be transported from the container through the isolator opening and into the collecting device.

Furthermore, the document FR 2 866 016 A1 discloses a method in which a container is emptied by gravity by tilting the container and generating a movable shaft by deforming a flexible wall (12) arranged at the bottom of the container. The wall of the container is held in place to prevent pocket formation. The container has an opening at one of its ends for attachment to a corresponding opening of a chamber. FR 2 866 016 A1 relates in particular to an application of a method for emptying bags containing sterile products in a chamber, and a device for emptying the contents of a container, for example a bag, in the chamber.

However, the known transfer systems still leave room for improvement, especially with regard to the structural design and the transfer of objects.

### SUMMARY

Against this background, it is an object of the present application to improve the structural design of a transfer system and a barrier system. Furthermore, it is an object of the present application to improve the transfer of objects in a transfer system and a barrier system.

According to a first aspect, a functional element for a beta container system is provided, wherein the functional element has a transfer surface for transferring the objects through a beta port opening of a beta port of a beta container of the beta container system, wherein the functional element can be arranged on the beta container in a defined arrangement, wherein the transfer surface is arranged at least partially in the beta port opening in the defined arrangement, and wherein a first bag section of a bag of the beta container is arranged in the defined arrangement between the beta port and the functional element.

According to a second aspect, a beta container system for a transfer system is provided, wherein the beta container system comprises a beta container and the functional element according to the first aspect, wherein the beta container has an internal volume, a bag enclosing the internal volume, and a beta port with a beta port opening, wherein the beta port can be coupled to an alpha port of the transfer system.

According to a third aspect, a transfer system for transferring the objects is provided, wherein the transfer system comprises an alpha port and the beta container according to the second aspect.

According to a fourth aspect, a transfer system for transferring the objects is provided, wherein the transfer system has an alpha port and a beta container, wherein the beta container has an internal volume, a bag enclosing the internal volume, and a beta port with a beta port opening, wherein the beta port can be coupled to an alpha port of the transfer system, wherein the transfer system further has a support surface for the bag and a movement device, wherein the movement device is arranged between the beta port and the support surface, and wherein the movement device is configured to move the bag vertically.

According to a fifth aspect, a barrier system is provided, wherein the barrier system comprises the transfer system according to the third or fourth aspect. The barrier system can in particular be an isolator system with an isolator.

The isolator may have an interior space. The isolator may have an isolator wall that encloses or surrounds the interior space. The isolator wall separates the interior space from an external environment surrounding the isolator. The isolator is preferably an aseptic isolator. An aseptic isolator has a highly clean or sterile, i.e. germ-free, environment in the interior space.

The isolator can preferably be part of a filling system with several process and processing stations. The filling system can, for example, be a system for filling and closing containers with a pharmaceutical or cosmetic substance. The system may in particular comprise a filling station and at least one closing station.

The transfer system is used to transfer objects, in particular closure elements, into and/or out of the isolator. The transfer system comprises the alpha port and the beta container.

The alpha port can be located on the isolator wall of the isolator. The alpha port has an alpha port opening. The alpha port opening forms a passage through the isolator wall. In other words, the isolator is accessible from the outside through the alpha port opening. The alpha port opening can have any shape. For example, the alpha port opening can be circular, elliptical, or rectangular. In a preferred embodiment, the alpha port opening is circular.

The alpha port may also have an alpha port base body. The alpha port base body can preferably be ring-shaped. In particular, the alpha port base body can extend through the isolator wall. The alpha port base body has an inside and an outside. The inside faces the interior space. The outside faces the external environment. The outside is specifically a side of the alpha port facing the beta port. The alpha port base body may comprise the alpha port opening. In particular, the alpha port opening may be formed by a recess in the alpha port base body that extends from the inside to the outside of the alpha port base body. In particular, the alpha port base body surrounds the alpha port opening. The alpha port may have an alpha port flange on the outside. In particular, the alpha port base body can form the alpha port flange on the outside. The alpha port flange surrounds the alpha port opening.

The alpha port may also have a door. The door is located at the alpha port opening of the alpha port. The door is used to open and close the alpha port opening. Preferably, the door is arranged movably, in particular pivotably, on the alpha port base body of the alpha port. The door can be moved to open or close the alpha port opening. In the closed state, the door tightly seals the alpha port opening. For example, the alpha port can comprise a drive device for moving the door.

The beta container has an internal volume. For example, objects such as closure elements can be arranged in the internal volume. The beta container has a bag. The bag surrounds or encloses the internal volume. The bag may be a flexible bag, particularly a sterile bag. The bag may consist of plastics material. The bag may have a bag wall. The bag wall may be flexible or resilient. In particular, the bag wall can be a plastics film.

The beta container comprises the beta port. The beta port can be located on the bag of the beta container. In particular, the beta port is connected to the bag. The beta port has a beta port opening. The beta port opening forms a passage through the bag wall to the internal volume. In other words, the internal volume is accessible from the outside through the beta port opening. The beta port opening can have any shape. For example, the beta port opening can be circular, elliptical, or rectangular. In a preferred embodiment, the beta port opening is circular.

The beta port may also have a beta port base body. The beta port base body can preferably be ring-shaped. In particular, the beta port base body can extend into the internal volume. The beta port base body has an inside and an outside. The inside faces the interior volume. The outside faces the external environment. The outside is in particular a side of the beta port facing the alpha port. The beta port base body may comprise the beta port opening of the beta port. In particular, the beta port opening may be formed by a recess in the beta port base body that extends from the inside to the outside of the beta port base body. In particular, the beta port base body surrounds the beta port opening. The beta port may have a beta port flange on the outside. In particular, the beta port base body can form the beta port flange on the outside. The flange surrounds the beta port opening.

The beta port may also have a cover element. The cover element can be arranged on or in the beta port opening of the beta port. The cover element is used to cover or close the beta port opening. The cover element can be detachably coupled to the beta port base body or the beta port flange. When the cover element is coupled to the beta port base body or the beta port flange, the cover element tightly closes the beta port opening and completely covers it.

The alpha port can be coupled to the beta port. In a coupled state, the internal volume of the beta container is connected to the interior space of the isolator. In the coupled state, the alpha port and the beta port are coupled in such a way that the internal volume and the interior space are isolated from the external environment. In particular, a seal can be arranged between the alpha port and the beta port. In the coupled state, the beta port flange can be arranged on the alpha port flange. In particular, the seal can be arranged between the alpha port flange and the beta port flange.

The beta port opening and the alpha port opening are preferably the same size. In a preferred embodiment, the beta port opening and the alpha port opening are circular. In particular, the beta port opening and the alpha port opening can have the same diameter.

For coupling purposes, the alpha port and the beta port may, for example, have corresponding coupling elements, wherein one or more coupling elements of the alpha port can be coupled to one or more coupling elements of the beta port. The coupling elements can be arranged on the alpha port base body and the beta port base body or on the beta port flange and the alpha port flange.

The door and the cover element are also capable of being coupled. Only when the door and the cover element are coupled can the door and the cover element be moved together. In particular, they can be moved in such a way that the alpha port opening and the beta port opening can be opened and closed together. In particular, the door and the cover element can couple together when the alpha port and the beta port are coupled together. This allows the alpha port opening and the beta port opening to be opened and closed together when the alpha port and the beta port are coupled together.

For coupling purposes, the door and the cover element can, for example, have corresponding coupling elements, wherein one or more coupling elements of the door can be coupled to one or more coupling elements of the cover element. The coupling elements can, for example, form a bayonet lock.

Objects to be transferred into the isolator, such as closure elements, can be provided in the beta container. The objects can then be transferred or introduced into the isolator in the coupled state of the alpha port and the beta port.

When the alpha and beta ports are coupled and objects are transferred from the beta container to the isolator, the objects are transferred in a transfer direction through the alpha port opening and the beta port opening. In particular, the transfer direction may be parallel to a direction extending from the inside to the outside of the beta port.

The transfer system may also have a support surface for the bag. The support surface is located outside the isolator. The support surface can in particular be part of a support structure. For example, the support structure can be a carriage. The support structure can be designed such that the support surface can be moved vertically and/or inclined relative to the horizontal. When the bag rests on the support surface, the bag can thus be moved vertically or tilted relative to the horizontal using the support structure. By lifting the bag and/or tilting the bag towards the beta port, the objects to be transferred can be moved within the bag towards the beta port opening for transfer.

According to the first aspect, an additional functional element for the beta container can be provided. The functional element can be arranged on the beta container. The function element is used to improve the transfer of objects from the beta container. In particular, the functional element serves to improve the transfer of objects between the beta container and the isolator. The functional element has a transfer surface for transferring objects. The transfer surface is used to transfer the objects through the beta port opening. The transfer of the objects takes place in particular on or above the transfer surface. This causes the objects to be spaced from the beta port during transfer.

For this purpose, the functional element can be arranged on the beta container in a defined arrangement. The functional element is arranged at least partially within the beta port opening, in the defined arrangement. In particular, the transfer surface in the defined arrangement is arranged at least partially in the beta port opening. In a particularly preferred embodiment, the functional element and in particular the transfer surface extends through the beta port opening.

Furthermore, the bag of the beta container is arranged in the defined arrangement between the beta port and the functional element. In particular, a first bag section of the bag is arranged in the defined arrangement between the beta port and the functional element. The first bag section may be connected to the beta port or attached to the beta port. The first bag section can be arranged in the defined arrangement below the functional element, in particular below the transfer surface. In particular, the first bag section extends between the functional element and the beta port in the transfer direction.

In other words, the functional element is designed such that it can be arranged at least partially in the beta port opening and the bag then lies between the functional element and the beta port. In particular, in the defined arrangement, at least a part of the functional element extends into the beta port opening. The functional element is therefore located outside the internal volume in the defined arrangement. In particular, in the defined arrangement the functional element is arranged from the outside on the bag, and the bag separates the functional element from the internal volume of the beta container. In this case, the bag can enclose the part of the functional element that is located in the beta port opening. In particular, the bag wall of the bag in the defined arrangement can enclose the functional element in the beta port opening.

The functional element has a first axial end and a second axial end in an axial direction. The first axial end and the second axial end are arranged on opposite sides of the functional element in the axial direction. The axial direction runs parallel to the transfer direction of the objects in the defined arrangement. The transfer direction runs in particular from the internal volume of the beta container through the beta port opening. The first axial end and the second axial end are arranged in the defined arrangement on opposite sides of the functional element in the transfer direction.

The functional element may have a functional element body. The functional element body may have the transfer surface. In particular, the transfer surface can be a surface of the functional element body. The transfer surface can be arranged in the defined arrangement on an upper side of the functional element body. In particular, the functional element body can, for example, have a recess which is open towards the upper side of the functional element body, wherein the transfer surface is arranged in the recess.

The functional element may have a functional element flange. In particular, the functional element body can have or form the functional element flange. The functional element flange may preferably be arranged at the first axial end.

In order to bring the functional element into the defined arrangement, the functional element is first arranged from the outside on the bag of the beta container. In particular, the functional element is arranged so that the first axial end is oriented towards the beta port. The functional element is then inserted into the beta port opening. However, since the functional element is located outside the bag, the bag is partially pushed or folded into the beta port opening. In particular, as a result, the part of the functional element that extends into the beta port opening is enclosed by the bag wall.

The bag of the beta container is then arranged in the defined arrangement in the beta port opening such that the bag extends over the transfer surface, is folded around the first axial end of the functional element, and then extends below the functional element back out of the beta port opening. When the functional element is brought into the defined arrangement in this way, a part of the bag that is attached to the top of the beta port can also be partially folded into the beta port by the bag tension that acts on the bag when the functional element is pushed into the beta port opening.

Objects such as closure elements can be provided in the beta container, particularly in its internal volume. When the beta port is coupled to the alpha port, these objects can then be introduced into the isolator. For this purpose, said objects must be transferred through the beta port opening and the alpha port opening.

When transferring objects in a transfer system between a beta container and an isolator, it is known, among other things, that contact of the objects to be transferred with a so-called “ring of concern” should be avoided. The “ring of concern” includes, for example, the contact surfaces between the alpha port and the beta port, in particular a peripheral line of the seal between the alpha port and the beta port. It is particularly desirable to avoid contact of the objects with the beta port opening and the alpha port opening as far as possible or completely.

For example, when using sterile bags as beta containers, the prior art has previously involved the use of an internal tube that can be removed from the sterile bag when the alpha and beta ports are coupled, in order to bridge the “ring of concern”. However, the tube must be removed from the sterile bag using a gloved hand from inside the isolator. This means that additional manual operation is necessary, which can lead to the potential spread of germs through the glove.

Using the new functional element, the “ring of concern” can be bridged in a simple and safe way. According to the invention, the functional element is arranged such that at least a part of the functional element with the transfer surface extends into the beta port opening, wherein a part of the bag also extends into the beta port opening and separates the functional element from the internal volume of the beta container.

On the transfer path, the objects must then be transferred from the inner volume via the transfer surface and are thus kept away from the beta port. In this way, a passage is created in the beta port for the objects to be transferred, through which the objects to be transferred can be brought into the isolator without contact with the beta port. In particular, contact of the objects with the beta port opening and the alpha port opening, in particular with the “ring of concern”, can be avoided in this way. In addition, the objects are also protected from contact with the beta port by the bag folded into the beta port by the functional element. The new functional element according to the first aspect thus bridges the “ring of concern” without the need for manual operation.

According to the fourth aspect, a movement device may be additionally provided in the transfer system. The movement device also serves to improve the transfer of objects out of the beta container.

The movement device is arranged between the beta port and the support surface, in particular when the beta port is coupled to the alpha port. The movement device is designed to move the bag vertically. In particular, the movement device can move the bag vertically relative to the support surface and the beta port. For example, the movement device can move the bag up and/or down, in particular raise and/or lower it. In particular, the movement device can push the bag upwards between the beta port and the support surface. The term “vertical(ly)” refers here to the operating position of the transfer system, in particular the alpha port. The operating position of the alpha port may depend on the orientation of the isolator wall on which the alpha port is located.

To move the bag, the movement device can, for example, have a movement element that is vertically movable. The movement element can also be movable in other directions, for example horizontally and/or rotationally. To move the movement element, the movement device can have a drive device, for example a motor. To move the bag, the movement element can in particular be brought into contact with the bag in order to then move the bag vertically between the support surface and the beta port.

Within the bag, it can happen that the objects get stuck or block each other in front of the beta port. This is counteracted by moving the bag vertically between the beta port and the support surface. In this way, the transfer of objects from the internal volume through the beta port opening is improved.

In a preferred refinement of the transfer system of the fourth aspect, the transfer system may further comprise a functional element, wherein the functional element has a transfer surface for transferring the objects through the beta port opening of the beta port, wherein the functional element is arranged or can be arranged at least partially in the beta port opening.

The functional element serves to improve the transfer of objects from the internal volume of the beta container through the beta port opening. For this purpose, the functional element is arranged at least partially in the beta port opening. The transfer surface then serves to space the objects from the beta port during transfer. The functional element can, for example, be formed integrally with the beta port. Alternatively, the functional element can also be inserted into the beta port via the alpha port and arranged there. Alternatively, the functional element can also be inserted into the beta port opening from the outside and positioned there, the functional element in this case partially pushing or folding the bag into the beta port opening. If such a functional element is arranged in the beta port opening, it may happen that during the transfer of the objects, the objects get stuck on the functional element on the bag side and are thus blocked. This is the case in particular if the objects in the bag are partially arranged below the transfer surface of the functional element. By moving the movement device vertically, the objects are lifted, allowing even blocked objects to reach the transfer surface. This can prevent a build-up of objects in front of the functional element. The use of the movement device is therefore particularly advantageous if the transfer system has such a functional element.

In a further refinement of the transfer system of the fourth aspect, the functional element can be designed according to the functional element according to the first aspect.

In a further refinement of the transfer system of the third aspect, the transfer system may further comprise a support surface for the bag and a movement device, wherein the movement device is arranged between the beta port and the support surface, and wherein the movement device is configured to move the bag vertically.

In a further refinement of the aspects, a second bag section of the bag can be arranged in the defined arrangement on or above the transfer surface.

The second bag section thus runs above the functional element. In particular, the second bag section extends in the transfer direction along the transfer surface. This will allow the objects to be transferred to the second bag section. In other words, the second bag section is arranged on the transfer surface during the transfer and thus lies between the transfer surface and the objects.

In a further refinement of the aspects, the first bag section can be adjacent to the second bag section.

The part of the functional element that is arranged within the beta port opening or extends into the beta port opening from the outside is thereby surrounded, in the defined arrangement, by means of the first bag section and the second bag section.

In a further refinement of the aspects, the functional element, in particular the transfer surface, can have an open cross-sectional geometry.

In this case, the cross-sectional geometry defines the shape of the functional element or the transfer surface in a section perpendicular to the transfer direction or to the axial direction. The cross-sectional geometry can be convex. For example, the functional element can be half-shell-shaped. In this refinement, the functional element therefore does not have a closed cross-sectional geometry. In particular, the functional element in this refinement is not annular, cylindrical or funnel-shaped.

In a further refinement of the aspects, a cross-sectional geometry of the transfer surface can be substantially semicircular, semi-elliptical or U-shaped.

In this way, the objects are also guided laterally during transfer. This further increases safety against lateral contact with the beta port, in particular with the “ring of concern”, during the transfer. For example, the functional element can have a bottom and two side walls that form the transfer surface. The side walls may extend in the transfer direction or in the axial direction. The side walls can be adjacent to the bottom. In particular, the two side walls can be arranged perpendicular to the transfer direction on opposite sides of the bottom. In particular, the transfer surface can be formed at least at the first axial end of the functional element according to the cross-sectional geometry. At the second axial end, the transfer surface may be substantially flat. This makes it easier to transport the objects from the internal volume to the transfer surface.

In a further refinement of the aspects, the transfer surface can be inclined or chute-shaped.

This will facilitate the transfer of objects through the beta port opening. In particular, the transfer surface and/or the bottom of the functional element can be inclined in the transfer direction in the defined arrangement. As a result, the transfer surface or the bottom is arranged lower at the first axial end of the functional element than at the second axial end.

In a further refinement of the aspects, the functional element is capable of being coupled to the beta port or to an alpha port or to a wall of a barrier system in order to fix the functional element in the defined arrangement.

In this way, the functional element is held securely in the defined arrangement. In particular, the functional element can be attached to the beta port or to an alpha port or to a wall of a barrier system. The coupling or fastening can be carried out, for example, via a bayonet lock or by means of one or more fastening means, in particular screws. The alpha port is in particular the alpha port of the transfer system. The wall of the barrier system is in particular the wall on which the alpha port is located. The wall is preferably an isolating wall.

In a further refinement of the aspects, the functional element can have one or more coupling sections for fastening the functional element.

Each coupling section of the functional element is capable of being coupled to a corresponding coupling section of the beta port, the alpha port or the wall in order to secure the functional element. For example, each coupling section may be capable of being engaged with the corresponding coupling section, in particular by rotation, in order to establish the coupling.

In a further refinement of the aspects, the beta port may have a cover element for covering the beta port opening.

The beta port opening can be closed using the cover element. The cover element thus serves to tightly cover or close the beta port opening.

In a further refinement of the aspects, the beta port may have a sealing element for sealing the coupling between the alpha port and the beta port.

The sealing element can preferably be arranged on the outside of the beta port base body. In particular, the sealing element can be arranged on the beta port flange. In particular, the beta port base body can have a groove on the outside surrounding the beta port opening into which the sealing element is inserted. The sealing element can, for example, be a lip seal. In the coupled state, the sealing element can be arranged between the alpha port flange and the beta port flange. In this way, the internal volume of the beta container and the interior space of the isolator are sealed from the external environment of the isolator.

In a further refinement of the aspects, the bag can extend into the beta port opening in the defined arrangement.

In this way, contact with the beta port can be avoided.

In a further refinement of the aspects, the bag may have a bag wall, wherein the bag wall in the defined arrangement encloses the functional element in the beta port opening.

As previously explained, the bag wall can consist of a resilient or flexible material, in particular a plastics material. The bag wall separates the functional element from the internal volume. In particular, the bag wall extends between the beta port and the functional element to the first axial end of the functional element; is then folded back around the functional element body, and then extends above the transfer surface back out of the beta port opening.

In a further refinement of the aspects, the isolator may have an interior space and an isolator wall enclosing the interior space, wherein the alpha port is arranged on the isolator wall, wherein the beta port can be coupled to the alpha port from an external environment of the isolator.

Objects can be transferred between the beta container and the isolator when the alpha port and the beta port are coupled together.

In a further refinement of the aspects, the alpha port can be arranged on a vertical wall surface of the isolator wall.

The vertical wall surface in the isolator runs parallel to a vertical direction. When feeding objects, it has thus far been necessary for the alpha port to be located not on a vertical wall, but on a sloping ceiling wall, for example on a bay window, in order to enable object feeding. Using the functional element, it is now possible to use an alpha port located on a vertical wall for object feeding. The alpha port therefore does not have to be placed on a bay window. In particular, the functional element can be fastened to the wall surface of the isolator wall in order to fasten or hold the functional element in the defined arrangement.

In a further refinement of the aspects, the barrier system may further comprise a feed device in the interior space of the isolator, wherein the feed device can be arranged from the inside on the alpha port.

The feed device is used to feed objects, in particular closure elements, into the isolator. If the feed device is located at the alpha port, the objects from the beta container can be fed into the isolator through the alpha and beta ports via the feed device. The feed device can have a pipe or a chute for this purpose. The feed device may comprise an arm which is pivotably arranged on the isolator wall, preferably above the alpha port.

In a further refinement of the aspects, the feed device can comprise a pipe, wherein a first, open end of the pipe can be arranged at the alpha port, in particular wherein the pipe is cylindrical or elliptical.

In particular, the first end of the pipe may extend into the alpha port opening. The objects from the beta container can then be brought into the isolator via the pipe. The pipe has a first open end and a second open end. During transfer, the objects enter the pipe at the first, open end and then come out of the pipe at the second, open end.

In a further refinement of the aspects, the pipe of the feed device can be arranged on the alpha port such that the first end of the pipe is arranged on the functional element.

As a result, objects transferred from the internal volume of the beta container via the transfer surface go directly into the pipe of the feed device. This ensures that the objects do not come into contact with the “ring of concern”. In this case, it is possible that the pipe is arranged on the alpha port in such a way that the pipe extends into the alpha port opening and in particular up to the beta port. Alternatively, the functional element can also be arranged in the defined arrangement such that the functional element extends through the beta port opening and the alpha port opening.

In a further refinement of the aspects, the pipe of the feed device can be arranged on the alpha port in such a way that the pipe runs obliquely to a horizontal direction, in particular wherein the first end of the pipe has an elliptical or cylindrical shape.

In particular, in this case, the second end of the pipe is arranged lower in the vertical direction than the first end of the pipe. This allows objects to be fed to be moved through the pipe by means of gravity. If the alpha port is located on a vertical wall surface, the alpha port opening is oriented horizontally. In other words, the transfer direction in this case runs parallel to a horizontal direction. In order to ensure that the first end of the pipe can still be arranged appropriately on the functional element, the first end of the pipe can be cut off at an appropriate angle. For example, the first end of the pipe may be elliptical if the pipe is cylindrical. In particular, the cross-section of the transfer surface at the first axial end of the functional element can be adapted to the shape of the pipe at the first end of the pipe.

It is understood that the features mentioned above and those to be explained below can be used not only in the combination specified in each case, but also in other combinations or on their own without departing from the scope of the present invention.

### DRAWINGS

Embodiments of the disclosure are shown in the drawings and are described in more detail below with reference thereto. In the drawings:

FIG. 1 is an isometric view of a first embodiment of an isolator system;

FIG. 2 is a longitudinal sectional view of the isolator system of FIG. 1;

FIG. 3 is an isometric view of the isolator system from FIG. 1 with the feed device pivoted away;

FIG. 4 is an isometric view of a transfer system of the isolator system of FIG. 1;

FIG. 5 is an isometric view of a rear side of the transfer system of FIG. 4;

FIG. 6 is a longitudinal sectional view of the transfer system of FIG. 4;

FIG. 7 is an isometric view of an alpha port of the transfer system of FIG. 4;

FIG. 8 is an isometric view of a rear side of the alpha port of FIG. 7;

FIG. 9 is an isometric view of a beta container of the transfer system of FIG. 4;

FIG. 10 is a longitudinal sectional view of the beta container of FIG. 9 without the functional element;

FIG. 11 is an isometric view of the beta container from FIG. 9 with the functional element;

FIG. 12 is an isometric view of a functional element of the beta container of FIG. 9; and

FIG. 13 is an isometric view of a rear side of the functional element from FIG. 12;

FIG. 14 is a longitudinal sectional view of the functional element of FIG. 12;

FIG. 15 is a longitudinal sectional view of a second embodiment of an isolator system; and

FIG. 16 is a longitudinal sectional view of the isolator system of FIG. 15 with an inclined support surface.

### DETAILED DESCRIPTION

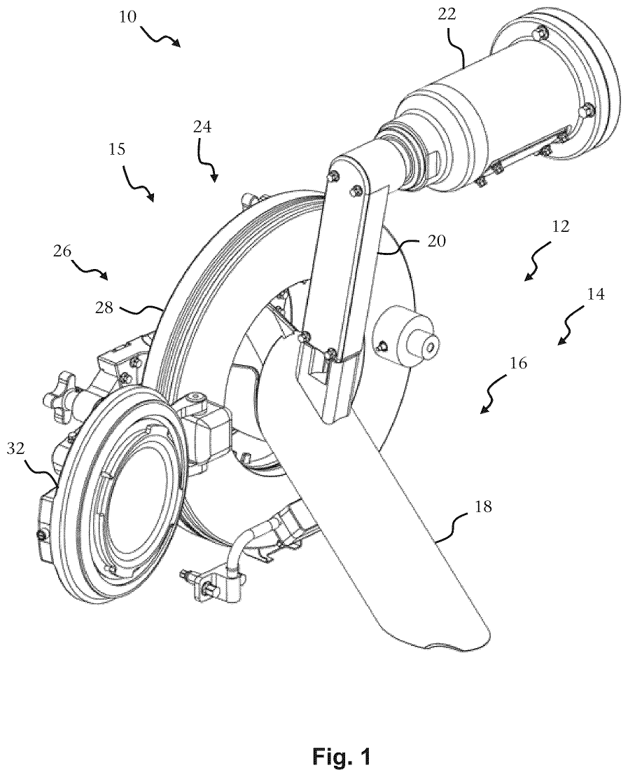

FIGS. 1 to 3 show a first embodiment of an isolator system as a barrier system, designated in its entirety by the reference sign 10.

The isolator system 10 has an isolator 12. The isolator 12 may have an interior space 14. The isolator may have an isolator wall (not shown) that encloses or surrounds the interior space 14. The isolator wall separates the interior space 14 from an external environment 15 surrounding the isolator 12. The isolator 12 is preferably an aseptic isolator.

The isolator system 10 may further comprise a feed device 16. The feed device 16 serves to feed objects into the interior space of the isolator 12. The objects can, for example, be closure elements, in particular plugs or closure caps. The feed device 16 is arranged in the interior space 14 of the isolator 12. The feed device 16 has a pipe 18. The feed device 16 has an arm 20. A first end of the arm 20 is fixedly connected to the pipe 18. A second end of the arm 20 is mounted on the isolator wall so as to be pivotable about a horizontal axis. The feed device 16 has a drive device 22. The drive device 22 is designed to pivot the arm 20 together with the pipe 18 about the horizontal axis. The pipe 18 has a first open end 82 and a second open end 84. The pipe 18 extends from the first end 82 to the second end 84. The pipe 18 is essentially cylindrical. The pipe 18 is cut obliquely at least at the first end 82. The pipe 18 is thus elliptical at the first end 82. The pipe 18 can also be cut obliquely at the two ends 82, 84. The pipe 18 is then elliptical at both ends 82, 84.

The isolator system 10 further comprises a transfer system 24. The transfer system 24 is used to transfer objects into the isolator or to transfer objects out of the isolator. In particular, objects such as closure elements can be introduced into the isolator via the transfer system 24.

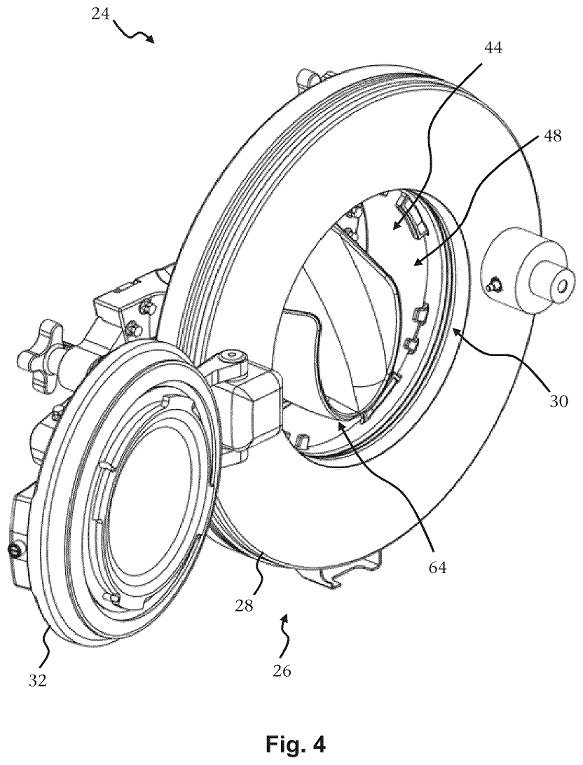

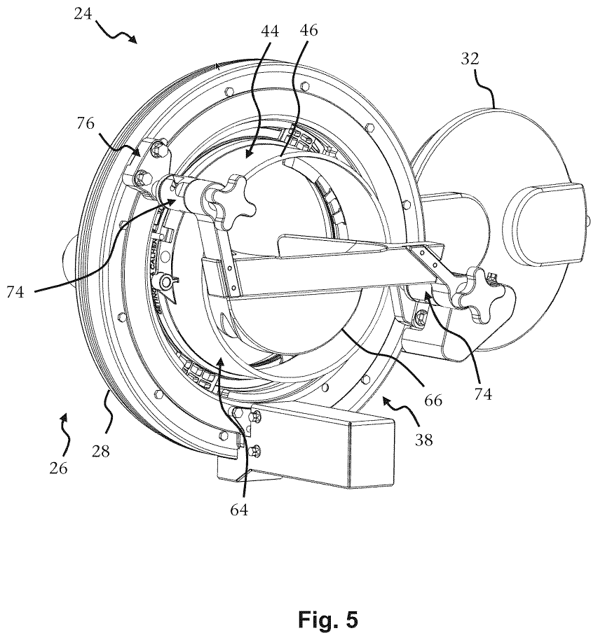

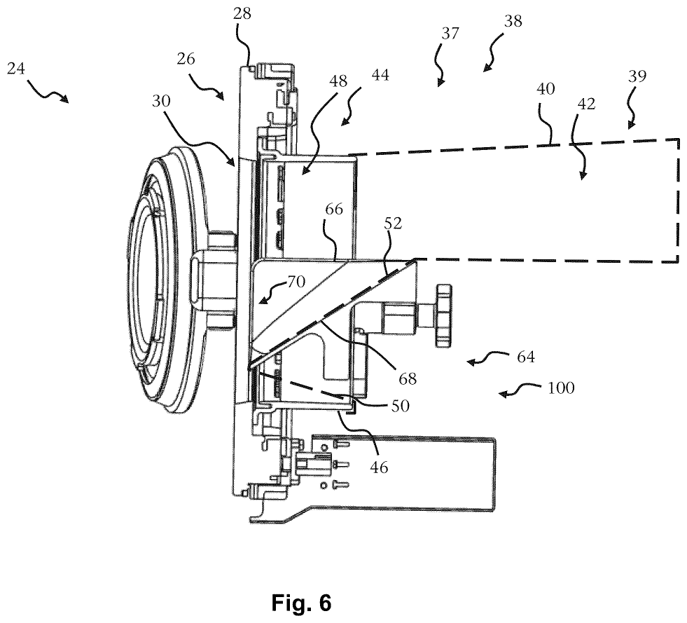

The transfer system 24 is shown in detail in FIGS. 4 to 6. The transfer system has an alpha port 26 and a beta container system 37. The beta container system 37 has a beta container 38. The alpha port 26 can be arranged on the isolator wall of the isolator 12. In particular, the alpha port 26 is arranged on a vertical wall surface of the isolator wall. The alpha port 26 forms in particular a closable passage through the isolator wall from the interior space 14 to the external environment 15.

The feed device 16 can be arranged from the inside on the alpha port 26. When the feed device 16 is arranged at the alpha port 26, the objects can be fed from the beta container 38 into the isolator 12 via the feed device 16.

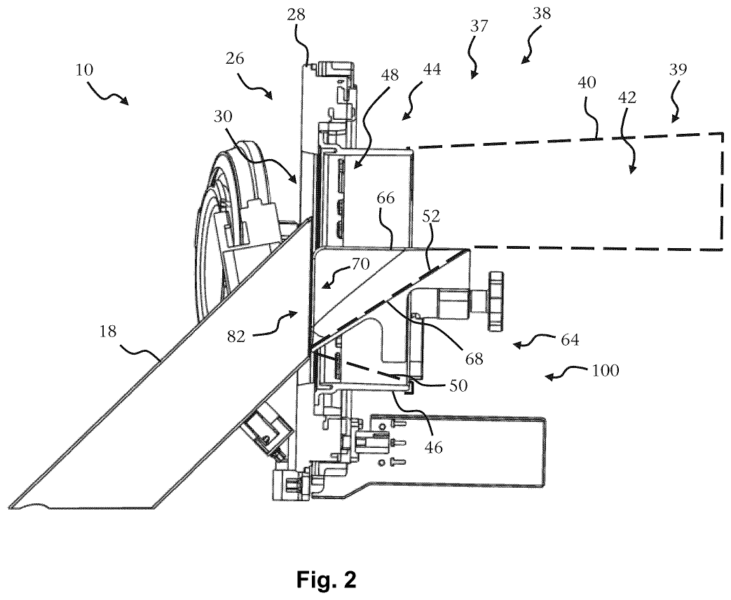

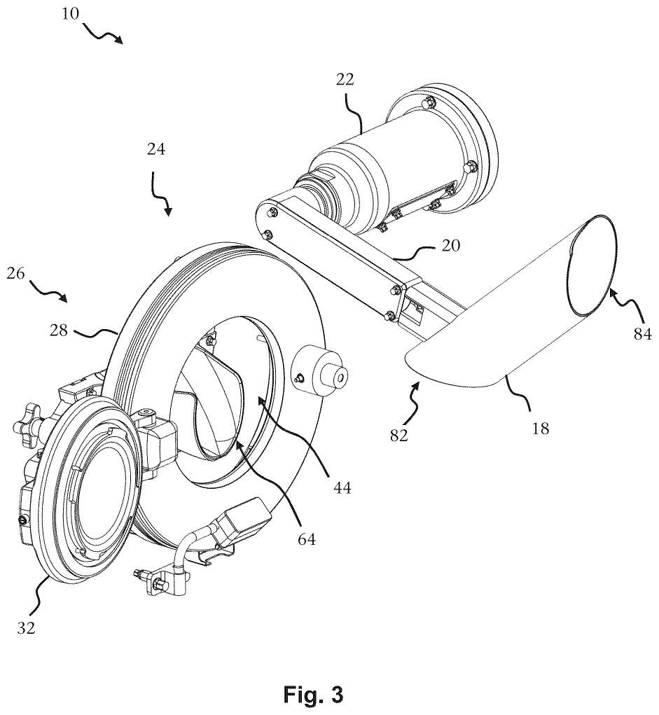

Preferably, the feed device 16 is arranged above the alpha port 26 so as to be pivotable in a vertical direction. The pipe 18 can be pivoted between a first position and a second position via the arm 20. In FIGS. 1 and 2, the pipe 18 is arranged in the first position. In the first position, the first end 82 of the pipe 18 is arranged at or in the alpha port 26. When the pipe 18 is arranged in the first position, objects from the beta container 38 can be inserted into the pipe 18. In FIG. 3, the pipe 18 is arranged in the second position. In the second position, the first end 82 of the pipe 18 is not arranged at the alpha port 26, but is moved away from it or spaced from the alpha port 26. When the pipe 18 is arranged in the second position, the door 32 can be opened and closed.

In the first position of the pipe 18, the first end 82 of the pipe 18 is arranged higher in the vertical direction than the second end 84. The pipe 18 therefore runs obliquely to a horizontal direction.

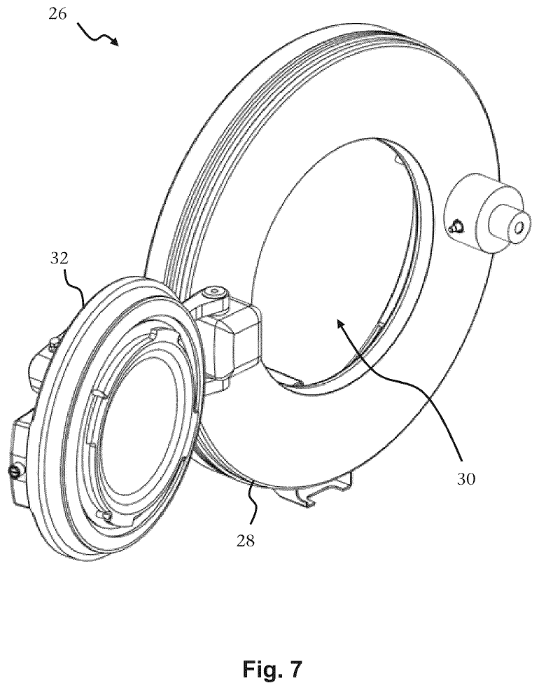

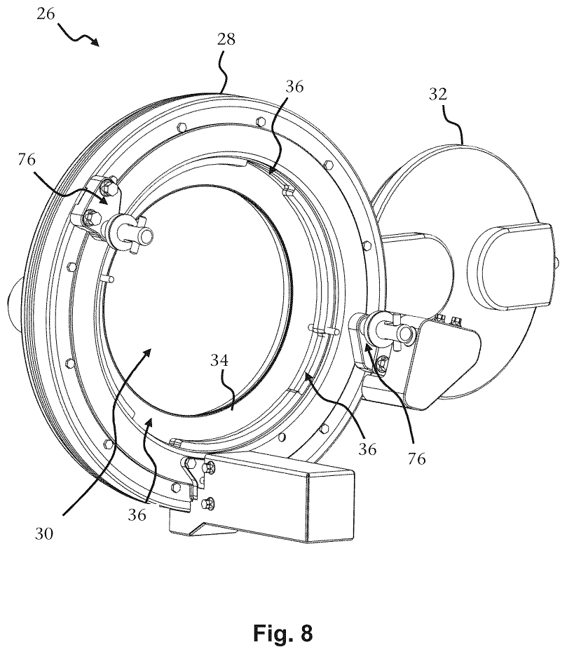

The alpha port 26 is shown in detail in FIGS. 7 and 8.

The alpha port 26 has an alpha port base body 28. The alpha port base body 28 can be ring-shaped. In particular, the alpha port base body 28 can extend through the isolator wall. The alpha port base body 28 has an inside and an outside. The inside faces the interior space. The outside faces the external environment 15. In the coupled state, the outside is in particular directed towards the beta container 38.

The alpha port 26 has an alpha port opening 30. The alpha port opening 30 forms a passage through the isolator wall. The alpha port opening 30 is circular. The alpha port opening 30 may be formed by a recess in the alpha port base body 28 that extends from the inside to the outside of the alpha port base body 28. The alpha port base body 28 surrounds the alpha port opening 30.

The alpha port 26 also has a door 32. The door 32 is located at the alpha port opening 30. The door 32 is used to open and close the alpha port opening 30. The door 32 is arranged movably, in particular pivotably, on the alpha port base body 28. The door 32 can be moved to open or close the alpha port opening 30. In a closed state, the door 32 tightly closes the alpha port opening 30. For example, the alpha port 26 may have a drive device (not shown) to move the door.

The alpha port 26 has an alpha port flange 34. The alpha port flange 34 is arranged on the outside of the alpha port base body 28. In particular, the alpha port base body 28 can form the alpha port flange 34 on the outside. The alpha port flange 34 surrounds the alpha port opening 30 on the outside.

The alpha port 26 may further comprise one or more coupling elements 36. The coupling elements 36 serve to couple the alpha port 26 to the beta container 38. The coupling elements 36 are arranged on the alpha port base body 28, in particular on the alpha port flange 34.

The alpha port 26 may further include one or more coupling sections 76. The coupling sections 76 serve to couple a functional element 64, which will be described below. The coupling sections 76 are arranged on the outside of the alpha port base body 28. In particular, the coupling sections 76 are arranged adjacent to the alpha port opening 30 and the alpha port flange 34.

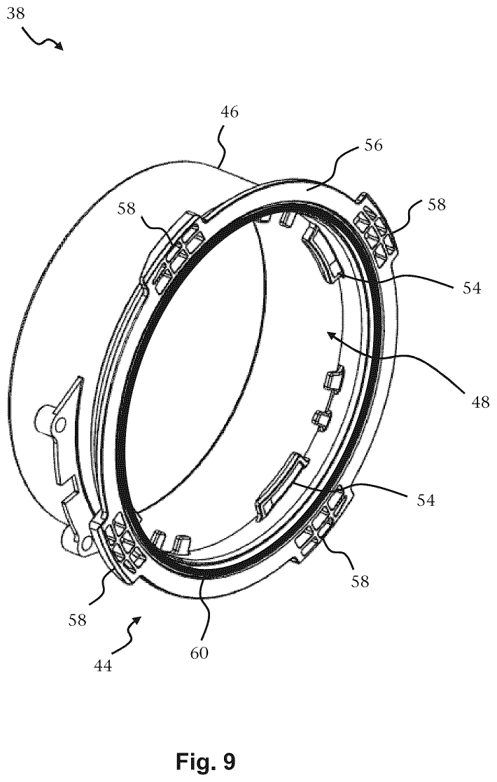

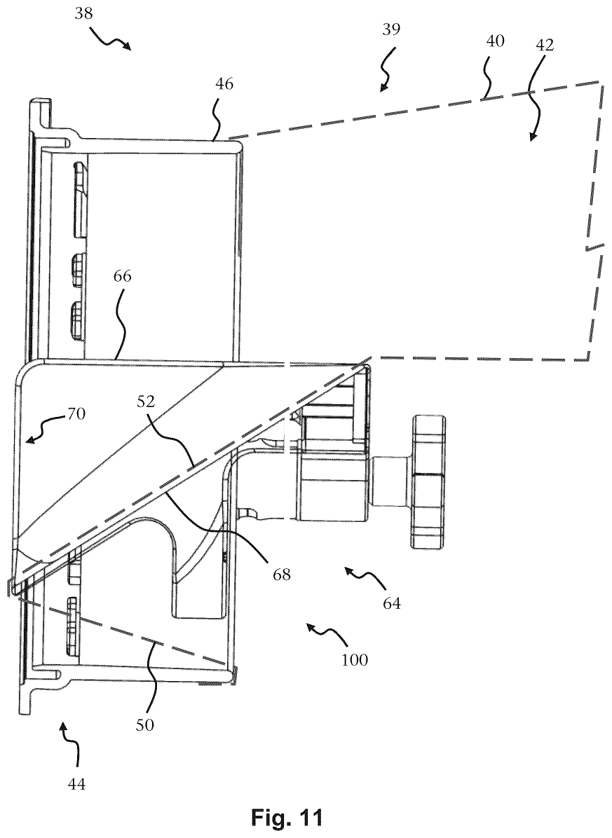

The beta container 38 is shown in detail in FIGS. 9 to 11.

The beta container 38 has a bag 39. The bag has a bag wall 40. The bag 39 can, for example, be designed to be flexible. In particular, the bag 39 may be a flexible bag. The bag 39 may consist of plastics material. The bag 39 can, for example, be a sterile bag.

The beta container 38 further has an internal volume 42. For example, objects such as closure elements can be arranged in the internal volume 42. The bag 39 surrounds or encloses the internal volume 42. In particular, the bag 39 isolates the internal volume 42 from the external environment 15 surrounding the beta container 38. The bag 39 and the internal volume 42 are schematically sketched in FIGS. 2, 6, 10, 11, 15 and 16. In the remaining figures of the isolator system 10 of the first embodiment, the bag 39 and the internal volume 42 are not shown, for illustrative purposes.

The beta container 38 has a beta port 44. The beta port 44 can be arranged on the bag 39 of the beta container 38. In particular, the beta port 44 may be connected to the bag 39. The alpha port 26 and beta port 44 are capable of being coupled together. In particular, the beta port 44 can be coupled to the alpha port 26 from the external environment 15. In a coupled state of the alpha port 26 and the beta port 44, the internal volume 42 of the beta container 38 is connected to the interior space 14 of the isolator 12.

The beta port 44 has a beta port base body 46. The beta port base body 46 may preferably be annular. In particular, the beta port base body 46 may extend through the bag wall 40. Alternatively, the bag wall 40 can also be located on the inside of the beta port base body 46. The beta port base body 46 has an inside and an outside. The inside faces the internal volume 42. The outside faces the external environment 15. In the coupled state, the outside is in particular directed towards the alpha port 26.

The beta port 44 has a beta port opening 48. The beta port is connected to bag 39. The beta port opening 48 forms a passage through the bag wall 40. In other words, the internal volume 42 is accessible from the outside through the beta port opening 48. The beta port opening 48 is circular in shape. The beta port opening 48 may be formed by a recess in the beta port base body 46 that extends from the inside to the outside of the beta port base body 46. In particular, the beta port base body 46 surrounds the beta port opening 48. In the coupled state, the alpha port opening 30 and the beta port opening 48 are arranged concentrically to each other.

The beta port opening 48 and the alpha port opening 30 are essentially the same size. In particular, the beta port opening 48 and the alpha port opening 30 may have the same diameter.

The beta port 44 has a beta port flange 56. The beta port flange 56 is arranged on the outside of the beta port base body 46. In particular, the beta port base body 46 can form the beta port flange 56 on the outside. The beta port flange 56 surrounds the beta port opening 48 on the outside. When coupled, the alpha port flange 34 and the beta port flange 56 are in peripheral contact with each other.

The beta port 44 may further include a sealing element 60. The sealing element 60 serves to seal the coupling between the alpha port 26 and the beta port 44. The sealing element 60 can be arranged on the outside of the beta port base body 46. In particular, the sealing element 60 can be arranged on the beta port flange 56. In particular, the beta port flange 56 may have on the outside a groove surrounding the beta port opening 48, into which the sealing element 60 is inserted. The sealing element can, for example, be a lip seal. In the coupled state, the sealing element 60 is arranged between the alpha port flange 34 and the beta port flange 56. In this way, the internal volume 42 of the beta container 38 and the interior space 14 of the isolator 12 are sealed from the external environment 15.

The beta port 44 may have a cover element. The cover element can be arranged on or in the beta port opening 48. The cover element serves to cover or close the beta port opening 48, in particular when the beta container 38 is not coupled to the alpha port.

When the cover element is arranged on or in the beta port opening 48 and covers the beta port opening 48, the beta container 38 is closed. The beta container 38 is then in a closed state.

If the cover element is not arranged on or in the beta port opening 48 and does not cover the beta port opening 48, in particular if the cover element is removed from the beta port opening 48, the beta container 38 is open. The beta container 38 is then in an open state. In FIGS. 1 to 6 and 9 to 11, the beta container 38 is shown in the open state without the cover element.

The cover element can be detachably coupled to the beta port base body 46 or the beta port flange 56. When the cover element is coupled to the beta port base body 46 or the beta port flange 56, the cover element closes the beta port opening 48. The cover element and the beta port base body 46 can have corresponding coupling elements for coupling. In particular, the beta port base body 46 can have projections 54 in the beta port opening 48 and the cover element can have corresponding receptacles for the projections 54. To secure the cover element to or in the beta port opening 48, the projections 54 can be brought into engagement with the receptacles.

The beta port 44 may further comprise one or more coupling elements 58. The coupling elements 58 serve to couple the beta port 44 with the alpha port 26. In particular, the coupling elements 58 of the beta port can couple to the coupling elements 36 of the alpha port to couple the alpha port and the beta port together. The coupling elements 58 are arranged on the beta port base body 46, in particular on the beta port flange 56.

The door 32 and the cover element can be coupled together. When the door 32 and the cover element are coupled, the door 32 and the cover element can be moved together. In particular, they can be moved in such a way that the alpha port opening 30 and the beta port opening 48 can be opened and closed together. In particular, the door 32 and the cover element can couple together when the alpha port 26 and the beta port 44 are coupled together. This allows the alpha port opening 30 and the beta port opening 48 to be opened and closed together when the alpha port 26 and the beta port 44 are coupled together.

For coupling, the door 32 and the cover element can have corresponding coupling elements, wherein one or more coupling elements of the door 32 can be coupled to one or more coupling elements of the cover element.

Objects, such as closure elements, which are to be transferred into the isolator 12, can be provided in the beta container 38. The objects can then be transferred or introduced into the isolator 12 in the coupled state of the alpha port 26 and the beta port 44. In particular, the objects can be fed from the beta container 38 through the beta port 44, the alpha port 26 and the feed device 16 into the isolator.

When objects are transferred from the beta container 38 to the isolator 12 in the coupled state of the alpha and beta ports 26, 44, the objects are in each case transferred in a transfer direction through the alpha port opening 30 and the beta port opening 48. In particular, the transfer direction may be parallel to a direction extending from the inside to the outside of the beta port 44. In particular, the transfer direction extends from the internal volume 42 through the beta port opening 48.

The beta container system 37 further comprises a functional element 64. The beta container 38 and the functional element 64 together form a beta container system 37. The functional element 64 is used to guide the transfer of objects between the beta container and the isolator. For transferring the objects, the functional element 64 has a transfer surface 68. The transfer surface 68 is used to transfer the objects through the beta port opening 48.

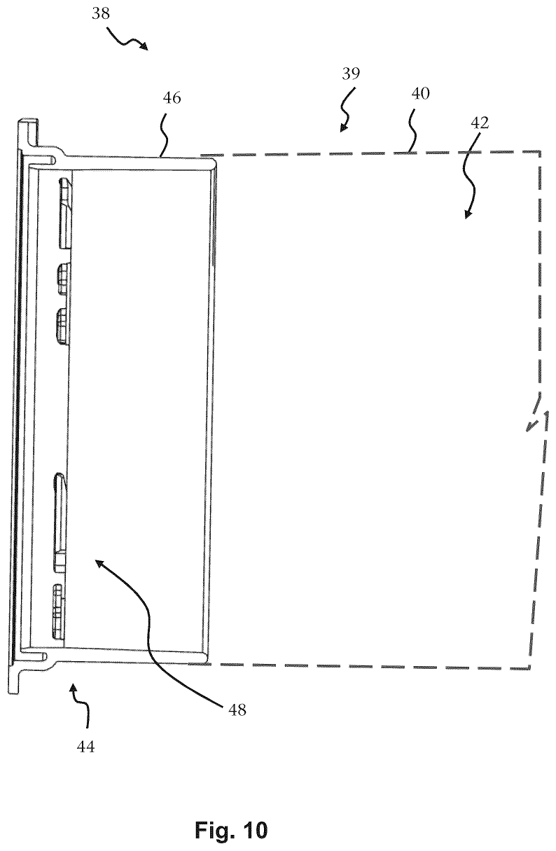

To transfer the objects through the beta port opening 48, the functional element 64 can be arranged on the beta container 38 in a defined arrangement 100. In FIGS. 1 to 6 and 11, the functional element 64 is shown in the defined arrangement 100. In FIGS. 9 and 10, the beta container 38 is shown without the functional element 64.

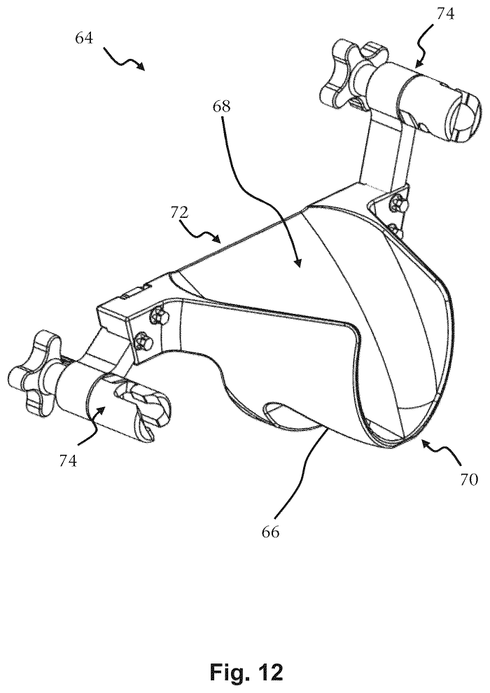

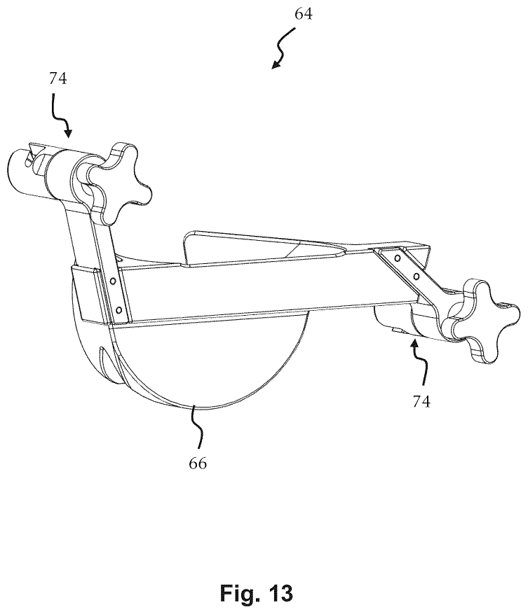

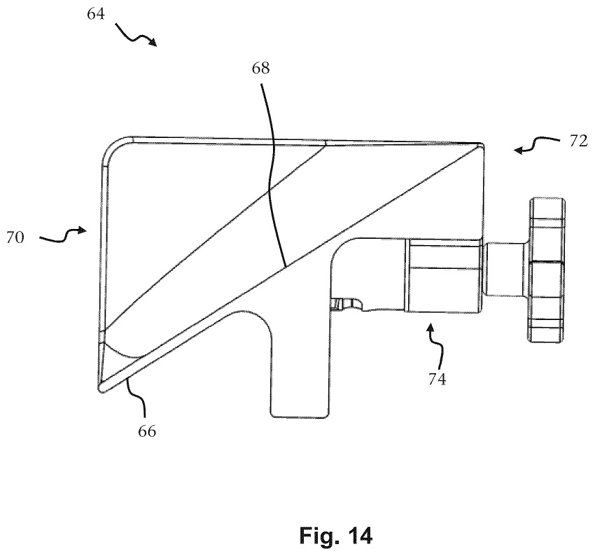

The functional element 64 is shown in detail in FIGS. 12 to 14.

The functional element 64 has a first axial end 70 and a second axial end 72 in an axial direction. The first axial end and the second axial end are arranged on opposite sides of the functional element 64 in the axial direction. In the defined arrangement 100, the axial direction runs parallel to the transfer direction of the objects. In the defined arrangement 100, the first axial end 70 and the second axial end 72 are arranged on opposite sides of the functional element 64 in the transfer direction. When the beta port 44 is coupled to the alpha port 26, the first axial end 70 faces the alpha port 26 and the second axial end 72 faces away from the alpha port 26.

The functional element 64 may have a functional element body 66. The functional element body 66 has the transfer surface 68. The transfer surface 68 is a surface of the functional element body 66. The transfer surface 68 is arranged in the defined arrangement 100 on an upper side of the functional element body 66. In particular, the functional element body 66 may have a recess which is open towards the upper side of the functional element body 66, wherein the transfer surface 68 is arranged in the recess.

The functional element 64 thus has an open cross-sectional geometry. In particular, the transfer surface 68 has an open cross-sectional geometry. The cross-sectional geometry is essentially convex. In particular, the functional element 64 is essentially half-shell-shaped, at least in the region of the transfer surface 68. The cross-sectional geometry of the transfer surface 68 can be substantially semicircular, semi-elliptical or U-shaped.

For example, the functional element 64 in the recess may have a bottom and two side walls that form the transfer surface 68. The side walls may extend in the transfer direction or in the axial direction. The side walls can be adjacent to the bottom. In particular, the two side walls can be arranged perpendicular to the transfer direction on opposite sides of the bottom.

In particular, the cross-sectional geometry of the transfer surface 68 can be convex, in particular semicircular, semi-elliptical or U-shaped, at least at the first axial end 70. The transfer surface 68 may be substantially flat at the second axial end 72.

The transfer surface 68 is preferably chute-shaped. In particular, the bottom of the transfer surface 68 may be inclined from the second axial end 72 toward the first axial end 70. As a result, the bottom is arranged lower at the first axial end 70 than at the second axial end 72.

The functional element 64 can be coupled to an alpha port 26 in order to fix the functional element 64 in the defined arrangement 100. The functional element 64 can thus be attached to the alpha port 26. For coupling, the functional element 64 has one or more coupling sections 74. The coupling sections 74 are arranged laterally next to the transfer surface 68. In particular, the coupling sections 74 are connected to the functional element body 66 at the second axial end 72. The coupling sections 74 serve to attach the functional element to the alpha port 26.

The coupling sections 74 of the functional element 64 can be coupled to the corresponding coupling sections 76 of the alpha port 26 in order to fasten the functional element 64. For example, each coupling portion 74 may be capable of being engaged with a corresponding coupling section 76, in particular by rotation, to establish the coupling.

In the defined arrangement 100, the functional element 64 is arranged at least partially within the beta port opening 48. The transfer surface 68 is arranged at least partially in the beta port opening 48 in the defined arrangement 100. In particular, the functional element 64 and in particular the transfer surface 68 in the defined arrangement 100 can extend through the beta port opening 48.

Furthermore, the bag 39 of the beta container 38 is arranged in the defined arrangement 100 between the beta port 44 and the functional element 64. In particular, the bag 39 surrounds the functional element 64 in the beta port opening 48. In the defined arrangement 100, a first bag section 50 is arranged between the beta port 44 and the functional element 64 and a second bag section 52 is arranged on or above the transfer surface 68. The first bag section 50 is adjacent to the second bag section 52. Thus, the functional element 64 in the beta port opening 48 is enclosed by the first bag section 50 and the second bag section 52.

The first bag section 50 is arranged in the defined arrangement 100 below the functional element 64. The first bag section 50 may be connected to the beta port 44 or attached to the beta port 44, in particular on the outside or on the inside of the beta port 44. The first bag section 50 extends between the functional element 64 and the beta port 44 in the transfer direction. In particular, the first bag section 50 may extend to the first axial end 70 of the functional element 64.

The second bag section 52 runs above the functional element 64. The second bag section 52 extends in the transfer direction along the transfer surface 68. In particular, the second bag section 52 may extend from the first axial end 70 to the second axial end 72 of the functional element 64.

In particular, the functional element 64 can only be arranged in the defined arrangement 100 when the beta port 44 and the alpha port 26 are coupled to one another. In FIG. 10, the beta container 38 is shown without the functional element 64. In order to bring the functional element 64 into the defined arrangement 100, the functional element 64 is first arranged from the outside on the bag 39, in particular from below on the bag 39. In particular, the functional element 64 is arranged such that the first axial end 70 is oriented towards the beta port 44 and the transfer surface 68 is oriented upwards. The functional element 64 is then inserted into the beta port opening 48. In this case, the bag 39 is partially pushed or folded into the beta port opening 38. In particular, the part of the functional element 64 that extends into the beta port opening 48 is thereby enclosed by the bag wall 40. This is shown in FIGS. 2, 6 and 11.

As previously described, objects, such as closure elements, can be provided in the internal volume 42 of the beta container 38, wherein these objects can then be introduced or fed into the interior space of the isolator 12 by means of the transfer system 24 and the feed device 16. For this purpose, the beta port 44 is first coupled to the alpha port 26 and the door 32 is opened together with the cover element. The pipe 18 of the feed device 16 can then be pivoted into the first position. This achieved state is shown in FIGS. 1 and 2. The objects can then be fed from the internal volume 42 through the beta port opening 48, the alpha port opening 30 and the pipe 18 into the isolator 12. In this case, the functional element 64 is arranged such that the objects are transferred via the transfer surface 68, in particular via the transfer surface 68 and the second bag section 52, through the beta port opening 48.

Since the alpha port 26 is arranged on a vertical wall surface of the isolator wall, the alpha port opening 30 extends in a horizontal direction from the inside to the outside of the alpha port base body 28. In the first position, the pipe 18 is arranged obliquely to the horizontal direction. As a result, the pipe 18 is aligned obliquely to the alpha port opening 30 in the first position.

In the first position of the pipe 18, the first end 82 of the pipe 18 is arranged at or in the alpha port opening 30. In the defined arrangement 100, the first axial end 70 of the functional element 64 extends substantially to the first end 82 of the pipe 18. Between the first end 82 of the pipe 18 and the first axial end 70 of the functional element 64, the bag 39 runs around the functional element 64.

The transfer surface 68 is arranged at the first axial end 70 in alignment with the pipe 18. In particular, the cross-sectional geometry of the transfer surface 68 at the first axial end 70 is designed such that it is aligned with the elliptical cross-sectional profile of the first end 82 of the pipe 18. This allows the objects to pass directly from the transfer surface 68 into the pipe 18 during transfer in the transfer direction.

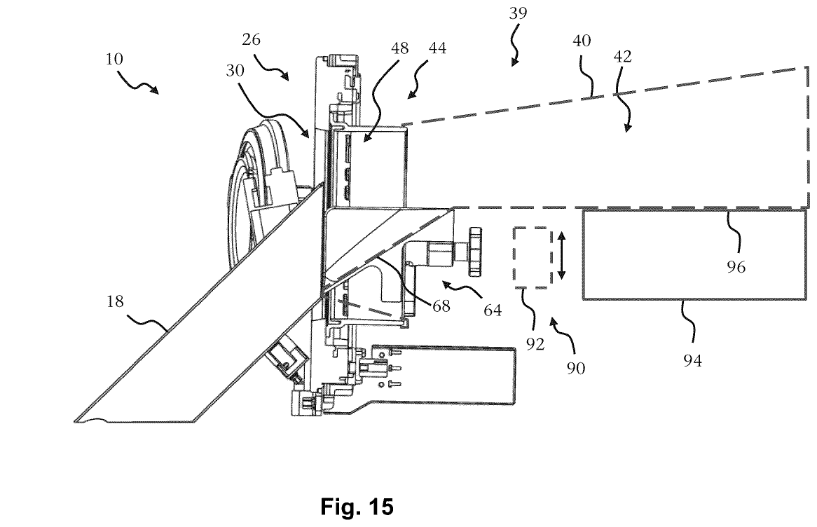

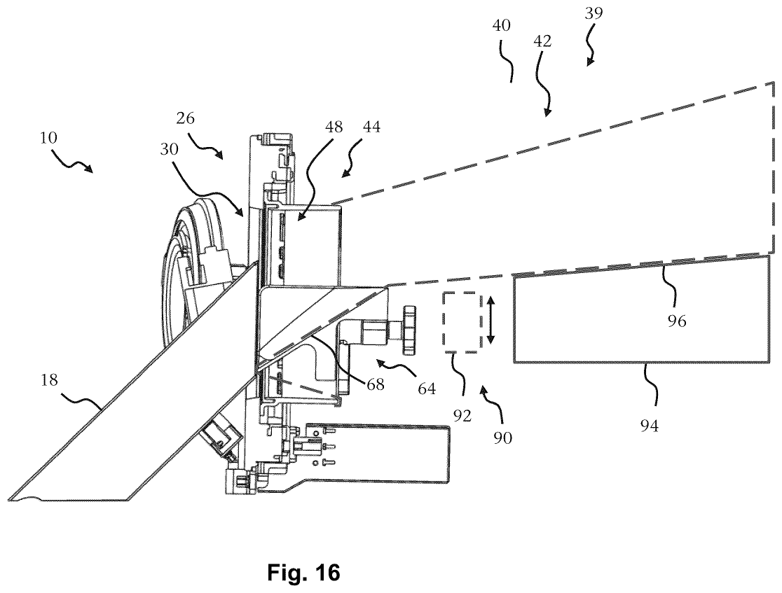

FIGS. 15 and 16 show a second embodiment of the isolator system 10. The isolator system 10 of the second embodiment corresponds essentially to the isolator system 10 of the first embodiment. Identical elements are identified by the same reference signs and will not be explained in more detail below.

In the second embodiment, the transfer system 24 further comprises a support surface 96 for the bag 39. The support surface 96 is arranged outside the isolator 12. The support surface 96 can in particular be part of a support structure 94. The support structure can be designed such that the support surface can be moved vertically and/or inclined relative to the horizontal. When the bag 39 rests on the support surface 96, the bag 39 can thus be moved vertically and/or inclined relative to the horizontal by means of the support structure 94.

In FIG. 15, the support surface 96 is shown flat, i.e. horizontally aligned. In FIG. 16, the support surface 96 is shown inclined. In particular, the support surface 96 can be inclined in the transfer direction, i.e. in the direction of the beta port 44, for transferring the objects.

In the second embodiment, the transfer system 24 may further comprise a movement device 90. The movement device 90 is arranged between the beta port 44 and the support surface 96, in particular when the beta port 44 is coupled to the alpha port 26. The movement device 90 is configured to move the bag 39 vertically. In particular, the movement device 90 can move the bag 39 vertically relative to the support surface 96 and the beta port 44.

To move the bag 39, the movement device 90 may have a movement element 92 that is vertically movable. Additionally, the movement element 92 is also capable of being moved in other directions, for example horizontally and/or rotationally. To move the movement element 92, the movement device 90 can have a drive device, for example a motor. To move the bag 39, the movement element 92 can in particular be brought into contact with the bag in order then to move the bag 39 vertically between the support surface 96 and the beta port 44.

The support structure 94 may further comprise a holding device which serves to hold or clamp the bag 39. If the bag 39 is clamped or held by means of the holding device and the bag is lifted and/or tilted by means of the support surface 96, the bag can be stretched or tensioned.

It is to be understood that the foregoing is a description of one or more preferred exemplary embodiments of the invention. The invention is not limited to the particular embodiment(s) disclosed herein, but rather is defined solely by the claims below. Furthermore, the statements contained in the foregoing description relate to particular embodiments and are not to be construed as limitations on the scope of the invention or on the definition of terms used in the claims, except where a term or phrase is expressly defined above. Various other embodiments and various changes and modifications to the disclosed embodiment(s) will become apparent to those skilled in the art. All such other embodiments, changes, and modifications are intended to come within the scope of the appended claims.

As used in this specification and claims, the terms “for example,” “e.g.,” “for instance, “such as,” and “like,” and the verbs “comprising,” “having,” “including,” and their other verb forms, when used in conjunction with a listing of one or more components or other items, are each to be construed as open-ended, meaning that the listing is not to be considered as excluding other, additional components or items. Other terms are to be construed using their broadest reasonable meaning unless they are used in a context that requires a different interpretation.

1. A functional element for a beta container system for transferring objects, comprising a transfer surface for transferring the objects through a beta port opening of a beta port of a beta container of the beta container system, wherein the functional element can be arranged on the beta container in a defined arrangement; the transfer surface is arranged at least partially in the beta port opening in the defined arrangement; and a first bag section of a bag of the beta container is arranged in the defined arrangement between the beta port and the functional element.

2. The functional element according to claim 1, wherein a second bag section of the bag is arranged in the defined arrangement on the transfer surface.

3. The functional element according to claim 2, wherein the first bag section is adjacent to the second bag section.

4. The functional element according to claim 1, wherein the functional element has an open cross-sectional geometry.

5. The functional element according to claim 1, wherein a cross-sectional geometry of the transfer surface is substantially semicircular, semi-elliptical or U-shaped.

6. The functional element according to claim 1, wherein the transfer surface is inclined or chute-shaped.

7. The functional element according to claim 1, wherein the functional element can be coupled to the beta port or to an alpha port or to a wall of a barrier system in order to fix the functional element in the defined arrangement.

8. The functional element according to claim 7, wherein the functional element has one or more coupling sections for fastening the functional element.

9. A beta container system for a transfer system, comprising a beta container and a functional element, wherein the functional element has a transfer surface for transferring objects through a beta port opening of a beta port of the beta container of the beta container system; the functional element can be arranged on the beta container in a defined arrangement; the transfer surface is arranged at least partially in the beta port opening in the defined arrangement; a first bag section of a bag of the beta container is arranged in the defined arrangement between the beta port and the functional element; the beta container has an internal volume, a bag which encloses the internal volume, and a beta port with a beta port opening; and the beta port can be coupled to an alpha port of the transfer system.

10. The beta container system according to claim 9, wherein the bag in the defined arrangement extends into the beta port opening.

11. The beta container system according to claim 9, wherein the bag has a bag wall, the bag wall in the defined arrangement encloses the functional element in the beta port opening.

12. A transfer system for transferring objects, comprising an alpha port and a beta container system, wherein the beta container system comprises a beta container and a functional element; the functional element has a transfer surface for transferring the objects through a beta port opening of a beta port of the beta container of the beta container system; the functional element can be arranged on the beta container in a defined arrangement; the transfer surface is arranged at least partially in the beta port opening in the defined arrangement; a first bag section of a bag of the beta container is arranged in the defined arrangement between the beta port and the functional element; the beta container has an internal volume, a bag which encloses the internal volume, and a beta port with a beta port opening; and the beta port can be coupled to the alpha port of the transfer system.

13. A transfer system for transferring objects, comprising an alpha port and a beta container, wherein the beta container has an internal volume, a bag which encloses the internal volume, and a beta port with a beta port opening; the beta port can be coupled to the alpha port of the transfer system; the transfer system further has a support surface for the bag and a movement device; the movement device is arranged between the beta port and the support surface; and the movement device is configured to move the bag vertically.

14. The transfer system according to claim 13, wherein the transfer system comprises a functional element, the functional element has a transfer surface for transferring the objects through the beta port opening of the beta port, and the functional element is arranged or can be arranged at least partially in the beta port opening.

15. The transfer system according to claim 14, wherein the functional element can be arranged on the beta container in a defined arrangement, the transfer surface is arranged at least partially in the beta port opening in the defined arrangement, and a first bag section of the bag of the beta container is arranged in the defined arrangement between the beta port and the functional element.

16. A barrier system, comprising a transfer system, wherein the transfer system comprises an alpha port and a beta container system; the beta container system comprises a beta container and a functional element; the functional element has a transfer surface for transferring objects through a beta port opening of a beta port of the beta container of the beta container system; the functional element can be arranged on the beta container in a defined arrangement; the transfer surface is arranged at least partially in the beta port opening in the defined arrangement; a first bag section of a bag of the beta container is arranged in the defined arrangement between the beta port and the functional element; the beta container has an internal volume, a bag which encloses the internal volume, and the beta port with the beta port opening; and the beta port can be coupled to the alpha port of the transfer system.

17. The barrier system according to claim 16, wherein the barrier system comprises an isolator, the isolator has an interior space and an isolator wall enclosing the interior space, the alpha port is arranged on the isolator wall, and the beta port can be coupled to the alpha port from an external environment of the isolator.

18. The barrier system according to claim 17, wherein the alpha port is arranged on a vertical wall surface of the isolator wall.

19. The barrier system according to claim 17, wherein the barrier system further comprises a feed device in the interior space of the isolator, the feed device can be arranged from the inside on the alpha port.

20. The barrier system according to claim 19, wherein the feed device comprises a pipe, and a first open end of the pipe can be arranged on the alpha port.

21. The barrier system according to claim 20, wherein the pipe of the feed device can be arranged on the alpha port such that the first open end of the pipe is arranged on the functional element.

22. The barrier system according to claim 20, wherein the pipe of the feed device can be arranged on the alpha port such that the pipe runs obliquely to a horizontal direction, and the first open end of the pipe has an elliptical shape.

23. The barrier system according to claim 16, wherein the barrier system is an isolator system.

24. A barrier system, comprising a transfer system, wherein the transfer system has an alpha port and a beta container; the beta container has an internal volume, a bag which encloses the internal volume, and a beta port with a beta port opening; the beta port can be coupled to the alpha port of the transfer system; the transfer system further has a support surface for the bag and a movement device; the movement device is arranged between the beta port and the support surface; and the movement device is configured to move the bag vertically.

25. The barrier system according to claim 24, wherein the barrier system comprises an isolator, the isolator has an interior space and an isolator wall enclosing the interior space, the alpha port is arranged on the isolator wall, and the beta port can be coupled to the alpha port from an external environment of the isolator.

26. The barrier system according to claim 25, wherein the alpha port is arranged on a vertical wall surface of the isolator wall.

27. The barrier system according to claim 24, wherein the barrier system further comprises a feed device in the interior space of an isolator, wherein the feed device can be arranged from the inside on the alpha port.

28. The barrier system according to claim 27, wherein the feed device comprises a pipe, a first open end of the pipe can be arranged on the alpha port, and the pipe is cylindrical or elliptical.

29. The barrier system according to claim 28, wherein the pipe of the feed device can be arranged on the alpha port such that the first open end of the pipe is arranged on the functional element.

30. The barrier system according to claim 28, wherein the pipe of the feed device can be arranged on the alpha port such that the pipe runs obliquely to a horizontal direction, and the first open end of the pipe has an elliptical shape.

31. The barrier system according to claim 24, wherein the barrier system is an isolator system.
